(19) United States
(12) Patent Application Publication
(10) Pub. No.: US 2024/0116961 A1
(43) Pub. Date: Apr. 11, 2024

(54) TRIPHENYPHOSPHINE MODIFIED SERTRALINE DERIVATIVE AND METHODS THEREOF

(71) Applicant: XIAMEN UNIVERSITY, Xiamen City (CN)

(72) Inventors: Shuaidong HUO, Xiamen City (CN); Jingbo QIN, Xiamen City (CN); Xiaohui CHEN, Xiamen City (CN); Xiaokun ZHANG, Xiamen City (CN)

(21) Appl. No.: 18/349,434

(22) Filed: Jul. 10, 2023

Related U.S. Application Data
(63) Continuation-in-part of application No. PCT/CN23/79290, filed on Mar. 2, 2023.

(30) Foreign Application Priority Data
Mar. 4, 2022 (CN) 202210214316.5

(51) Int. Cl. C07F 9/54; A61P 35/00

(52) U.S. Cl. CPC C07F 9/5442; A61P 35/00

(57) ABSTRACT

The present disclosure discloses a triphenylphosphine-modified sertraline derivative, a prepare method and an application thereof, a structural formula is as follows:

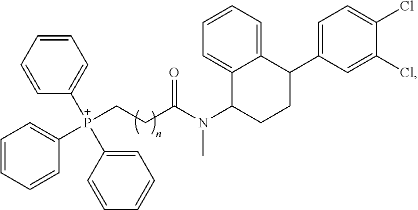

wherein n=1, 3, 5, 7, 9 or 11. The present disclosure can be used as an inducer for mitophagy for the prevention and/or treatment of mitophagy-related diseases including tumors and neurodegenerative diseases.

### RELATED APPLICATIONS

This application is a continuation-in-part of International patent application PCT/CN2023/079290, filed on Mar. 2, 2023, which claims priority to Chinese patent application 202210214316.5, filed on Mar. 4, 2022. International patent application PCT/CN2023/079290 and Chinese patent application 202210214316.5 are incorporated herein by reference.

### FIELD OF THE DISCLOSURE

The present disclosure relates to the field of pharmaceutical technologies of organic compounds, and specifically relates to a triphenylphosphine-modified sertraline derivative and methods thereof.

### BACKGROUND OF THE DISCLOSURE

The mitochondria are “energy factories” of the cells and are main organelles that produce and synthesize adenosine triphosphate (ATP) for cell survival and many other important cellular functions. Approximately 95% of energy required by the cells comes from the mitochondria. When the mitochondria are damaged, large amounts of reactive oxygen species (ROS) of the mitochondria are released into the cells, causing many diseases such as inflammation, cardiovascular disease, cancer, immune diseases, neurodegenerative diseases, and aging. The mitochondria regulate normal function of the whole cells and even the entire living organism by regulating the number, structure, and function of the mitochondria. Therefore, a healthy state of the cells can be achieved by maintaining normal function of the mitochondria, and diseases can be treated by killing and pro-apoptotic effects on non-normal cells (e.g., tumor cells) that are achieved by impairing or modulating mitochondrial function.

Mitophagy, a selective autophagy that removes damaged mitochondria, has been suggested as a possible mechanism for mitochondrial quality control to maintain intracellular environment balance and to prevent diseases such as aging and cancer. Recent reports have found that the mitophagy activates CD8⁺ T cells and NK cells to kill tumor cells by activating adaptive immunity and promoting tumor cell apoptosis, thus inhibiting tumor generation and development. At the same time, the mitophagy can inhibit the generation of inflammatory vesicles in a tumor microenvironment, suppress inflammation, and also inhibit tumor generation and development. However, by targeting the mitochondria, many unknowns still exist in drugs used to treat the tumors by inducing over-mitophagy.

### BRIEF SUMMARY OF THE DISCLOSURE

The present disclosure provides a triphenylphosphine-modified sertraline derivative to solve the deficiencies of the existing techniques.

The present disclosure provides a method for preparing the triphenylphosphine-modified sertraline derivative.

The present disclosure provides a method for using the triphenylphosphine-modified sertraline derivative.

A technical solution of the present disclosure is as follows.

A triphenylphosphine-modified sertraline derivative, a structural formula is as follows:

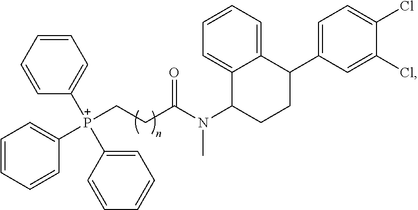

wherein n=1, 3, 5, 7, 9 or 11.

A preparation method of the triphenylphosphine-modified sertraline derivative, comprising:
(1) mixing p-triphenylphosphine, bromo organic acid, and a first organic solvent, and reacting by refluxing to obtain an intermediate, the bromo organic acid is 3-bromopropionic acid, 5-bromopentanoic acid, 7-bromoheptanoic acid, 9-bromononanoic acid, 11-bromoundecanoic acid, or 13-bromotridecanoic acid;
(2) mixing the intermediate obtained from the step (1), a second organic solvent, N-(3-dimethylaminopropyl)-N′-ethylcarbodiimide hydrochloride (EDCI), and 1-hydroxybenzotriazole (HOBt) to even under a room temperature condition, then adding sertraline hydrochloride and N,N-diisopropylethylamine, reacting at room temperature, then distilling under reduced pressure, and rotatably drying to obtain a crude product; and
(3) purifying the crude product obtained from the step (2) to obtain the triphenylphosphine-modified sertraline derivative.

In a preferred embodiment of the present disclosure, the first organic solvent is acetonitrile, and the second organic solvent is dichloromethane.

An application of the triphenylphosphine-modified sertraline derivative as an inducer for mitophagy.

An application of the triphenylphosphine-modified sertraline derivative for preparing a drug for at least one of prevention or treatment of a mitophagy-related disease.

In a preferred embodiment of the present disclosure, the mitophagy-related disease comprises tumors and neurodegenerative diseases.

A composition for at least one of prevention or treatment of a mitophagy-related disease, an active ingredient of the composition comprises the triphenylphosphine-modified sertraline derivative or a pharmaceutically acceptable salt, ester, prodrug, or hydrate thereof, and the mitophagy-related disease comprises tumors and neurodegenerative diseases.

In a preferred embodiment of the present disclosure, the active ingredient is the triphenylphosphine-modified sertraline derivative or the pharmaceutically acceptable salt, ester, prodrug, or hydrate thereof.

The present disclosure has the following advantages: the present disclosure can be used as an inducer for mitophagy for the prevention and/or treatment of mitophagy-related diseases, including tumors and neurodegenerative diseases.

### BRIEF DESCRIPTION OF THE DRAWING

FIG. 1 shows a graph of the experimental results of Embodiment 7 of the present disclosure.

FIGS. 2A-2D show graphs of the experimental results of Embodiment 8 of the present disclosure.

FIG. 3 shows a graph of the experimental results of Embodiment 9 of the present disclosure.

FIG. 4 shows a first graph of the experimental results of Embodiment 10 of the present disclosure.

FIG. 5 shows a second graph of the experimental results of Embodiment 10 of the present disclosure.

FIG. 6 shows a third graph of the experimental results of Embodiment 10 of the present disclosure.

FIG. 7 shows the hydrogen spectral data of Mito-2-Ser in Embodiment 1 of the present disclosure.

FIG. 8 shows the mass spectral data of the Mito-2-Ser in Embodiment 1 of the present disclosure.

FIG. 9 shows the hydrogen spectral data of Mito-4-Ser in Embodiment 2 of the present disclosure.

FIG. 10 shows the mass spectral data of the Mito-4-Ser in Embodiment 2 of the present disclosure.

FIG. 11 shows the hydrogen spectral data of Mito-6-Ser in Embodiment 3 of the present disclosure.

FIG. 12 shows the mass spectral data of the Mito-6-Ser in Embodiment 3 of the present disclosure.

FIG. 13 shows the hydrogen spectral data of Mito-8-Ser in Embodiment 4 of the present disclosure.

FIG. 14 shows the mass spectral data of the Mito-8-Ser in Embodiment 4 of the present disclosure.

FIG. 15 shows the hydrogen spectral data of Mito-10-Ser in Embodiment 5 of the present disclosure.

FIG. 16 shows the mass spectral data of the Mito-10-Ser in Embodiment 5 of the present disclosure.

FIG. 17 shows the hydrogen spectral data of Mito-12-Ser in Embodiment 6 of the present disclosure.

FIG. 18 shows the mass spectral data of the Mito-12-Ser in Embodiment 6 of the present disclosure.

### DETAILED DESCRIPTION OF THE EMBODIMENTS

The technical solution of the present disclosure will be further described below in combination with the drawings and the embodiments.

### Embodiment 1: Preparation of (3-(((1S,4S)-4-(3,4-dichlorophenyl)-1,2,3,4-tetrahydronaphthalen-1-yl)(methyl)amino)-3-propanoyl)triphenylphosphine (Mito-2-Ser)

A synthetic route for this embodiment is as follows:

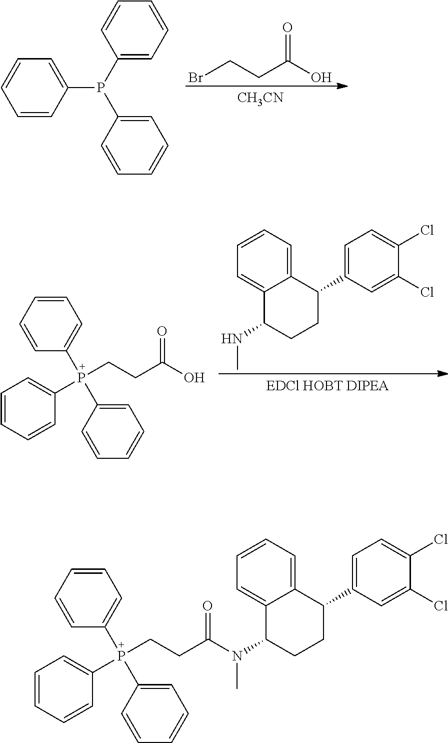

It specially comprises the following steps:
(1) p-triphenylphosphine (1.00 g, 3.81 mmol), 3-bromopropanoic acid (0.59 g, 3.81 mmol), and acetonitrile are sequentially added into a dried 250 mL two-necked flask. The reaction is refluxed for 9 hours, and the reaction is stopped after measurements using thin-layer chromatography (TLC) indicate that the reaction is complete. The acetonitrile is removed by reduced-pressure distillation, and 2-(carboxyethyl)triphenylphosphonium bromide as an intermediate is obtained by crystallization for purification.
(2) Dichloromethane (10 mL), the 2-(carboxyethyl)triphenylphosphonium bromide (100 mg, 0.24 mmol), N-(3-dimethylaminopropyl)-N′-ethylcarbodiimide hydrochloride (EDCI) (92 mg, 0.48 mmol), and 1-hydroxybenzotriazole (HOBt) (33 mg, 0.24 mmol) are sequentially added into a dried 50 mL reaction vial at room temperature (e.g., 20-25° C.) and stirred at room temperature for 1 hour. Sertraline hydrochloride (82 mg, 0.24 mmol) is added while stirring, catalyzed by N,N-diisopropylethylamine (93 mg, 0.72 mmol), and reacted overnight at room temperature. The reaction is stopped after the measurements using TLC indicate that the reaction is complete. Then the reaction solution is distilled under reduced pressure and rotatably dried to obtain a crude product.
(3) The obtained crude product is separated by silica gel column chromatography (the eluent is petroleum ether:ethyl acetate=1:1, volume/volume (v/v)) to obtain 96 mg of a solid product of the Mito-2-Ser in a 52.7% yield.

The spectrum data of the Mito-2-Ser is as follows: ¹H nuclear magnetic resonance spectroscopy (NMR) (600 MHz, DMSO-d₆) δ7.88-7.93 (m, 3H), 7.82-7.88 (m, 6H), 7.77 (dt, J=3.21, 7.47 Hz, 5H), 7.65-7.74 (m, 1H), 7.51-7.55 (m, 1H), 7.17-7.30 (m, 3H), 6.73-7.02 (m, 3H), 4.28 (br. s., 1H), 3.76-3.94 (m, 2H), 2.60 (br. s., 2H), 2.46 (s, 1H), 2.14-2.30 (m, 2H), 1.61-1.68 (m, 1H), 1.48 (d, J=3.67 Hz, 1H), 1.38 (qd, J=7.36, 14.97 Hz, 1H), 1.24 (br. s., 2H); HRMS (ESI, m/z): calculated for C₃₈H₃₅Cl₂NOP⁺ [M]⁺ 622.1828, found 622.1821.

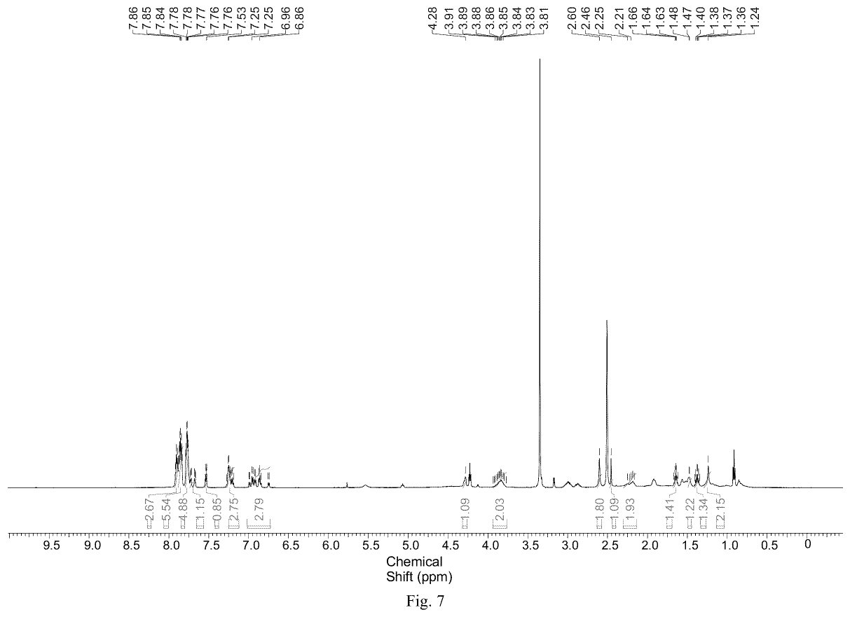

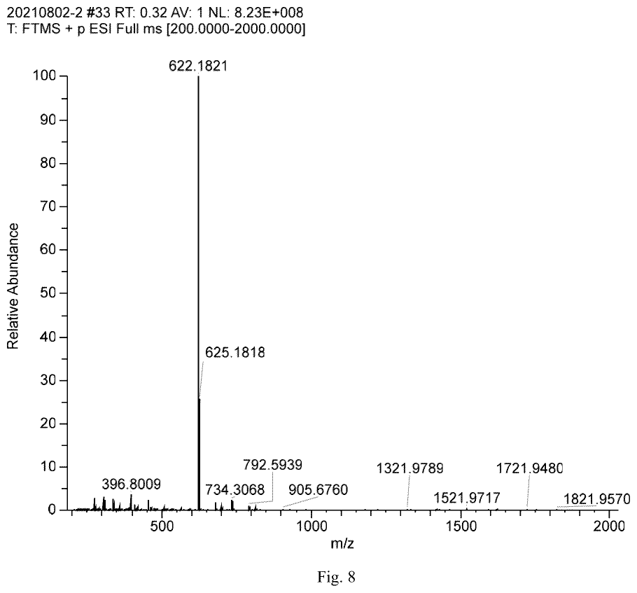

The hydrogen spectrum data of the Mito-2-Ser is shown in FIG. 7. The mass spectrum data of the Mito-2-Ser is shown in FIG. 8.

### Embodiment 2: Preparation of (5-(((1S,4S)-4-(3,4-dichlorophenyl)-1,2,3,4-tetrahydronaphthalen-1-yl)(methyl)amino)-5-oxopentyl)triphenylphosphine (Mito-4-Ser)

The preparation method is the same as in Embodiment 1, 5-bromopentanoic acid is used instead of 3-bromopropanoic acid in Embodiment 1, and the formula of the Mito-4-Ser is as follows:

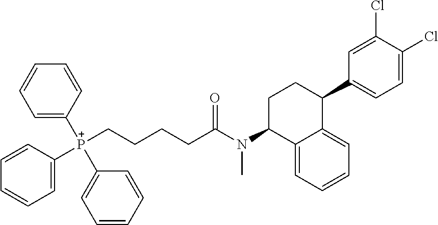 [SMILES: CN(C(=O)CCCC[P+](c1ccccc1)(c1ccccc1)c1ccccc1)[C@H]1CC[C@@H](c2ccc(Cl)c(Cl)c2)c2ccccc21]

The spectrum data of the Mito-4-Ser is as follows: ¹H NMR (600 MHz, DMSO-d₆) δ7.87-7.94 (m, 3H), 7.75-7.86 (m, 11H), 7.65-7.74 (m, 1H), 7.56 (d, J=8.25 Hz, 1H), 7.18-7.32 (m, 3H), 6.86-7.06 (m, 3H), 4.29-4.36 (m, 1H), 3.62-3.71 (m, 2H), 2.69 (br. s., 2H), 2.53-2.58 (m, 1H), 2.40-2.49 (m, 1H), 2.16-2.35 (m, 2H), 1.92-2.02 (m, 1H), 1.74-1.85 (m, 2H), 1.62-1.66 (m, 2H), 1.38 (qd, J=7.38, 14.90 Hz, 1H), 1.24 (br. s., 2H); HRMS (ESI, m/z): calculated for C₄₀H₃₉Cl₂NOP⁺ [M]⁺650.2141, found 650.2136.

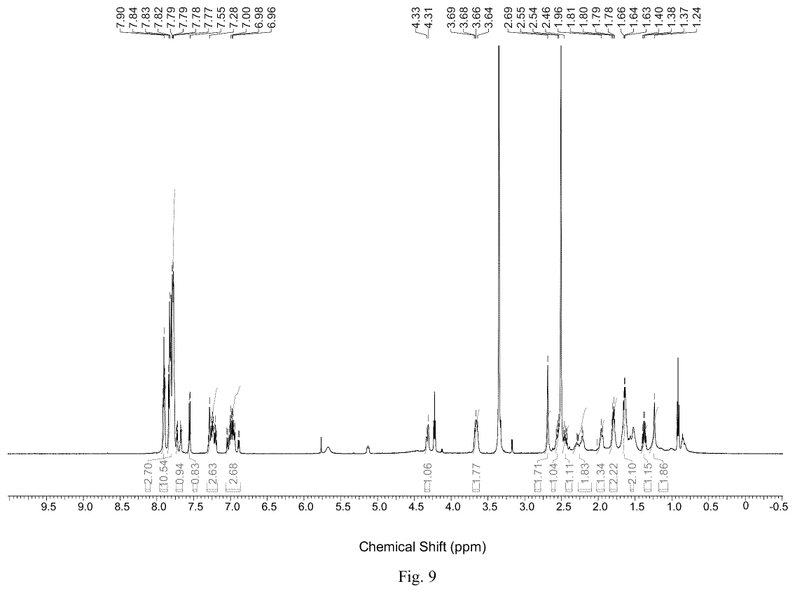

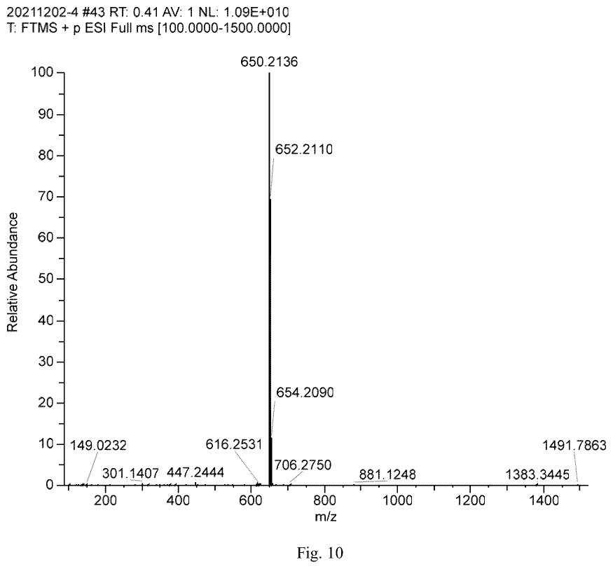

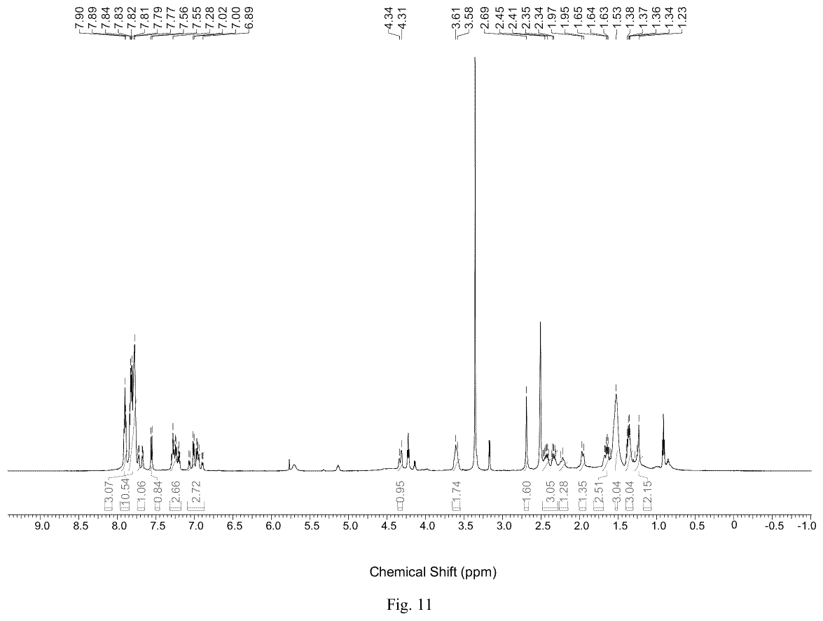

The hydrogen spectrum data of the Mito-4-Ser is shown in FIG. 9. The mass spectrum data of the Mito-4-Ser is shown in FIG. 10.

### Embodiment 3: Preparation of (7-(((1S,4S)-4-(3,4-dichlorophenyl)-1,2,3,4-tetrahydronaphthalen-1-yl)(methyl)amino)-7-oxoheptyl)triphenylphosphine (Mito-6-Ser)

The preparation method is the same as in Embodiment 1, 7-bromoheptanoic acid is used instead of 3-bromopropanoic acid in Embodiment 1, and the formula of the Mito-6-Ser is as follows:

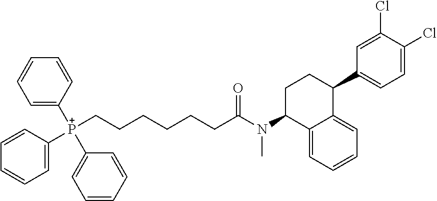 [SMILES: CN(C(=O)CCCCCC[P+](c1ccccc1)(c1ccccc1)c1ccccc1)[C@H]1CC[C@@H](c2ccc(Cl)c(Cl)c2)c2ccccc21]

The spectrum data of the Mito-6-Ser is as follows: ¹H NMR (600 MHz, DMSO-d₆) δ7.86-7.96 (m, 3H), 7.74-7.85 (m, 11H), 7.65-7.74 (m, 1H), 7.56 (d, J=8.44 Hz, 1H), 7.17-7.32 (m, 3H), 6.87-7.09 (m, 3H), 4.30-4.36 (m, 1H), 3.60 (d, J=13.57 Hz, 2H), 2.69 (br. s., 2H), 2.28-2.48 (m, 3H), 2.23 (d, J=13.57 Hz, 1H), 1.96 (d, J=13.20 Hz, 1H), 1.58-1.71 (m, 3H), 1.53 (br. s., 3H), 1.31-1.40 (m, 3H), 1.18-1.28 (m, 2H); HRMS (ESI, m/z): calculated for C₄₂H₄₃Cl₂NOP⁺ [M]⁺678.2454, found 678.2449.

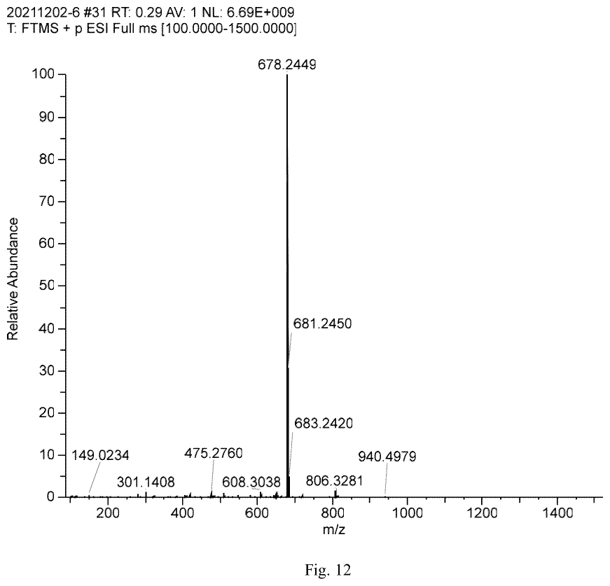

The hydrogen spectrum data of the Mito-6-Ser is shown in FIG. 11. The mass spectrum data of the Mito-6-Ser is shown in FIG. 12.

### Embodiment 4: Preparation of (9-(((1S,4S)-4-(3,4-dichlorophenyl)-1,2,3,4-tetrahydronaphthalen-1-yl)(methyl)amino)-9-oxononyl)triphenylphosphine (Mito-8-Ser)

The preparation method is the same as in Embodiment 1, 9-bromononanoic acid is used instead of 3-bromopropanoic acid in Embodiment 1, and the formula of the Mito-8-Ser is as follows:

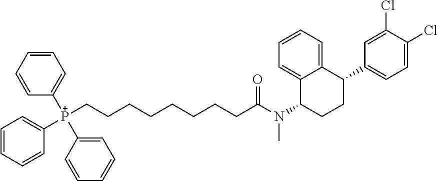 [SMILES: CN(C(=O)CCCCCCCC[P+](c1ccccc1)(c1ccccc1)c1ccccc1)[C@H]1CC[C@@H](c2ccc(Cl)c(Cl)c2)c2ccccc21]

The spectrum data of the Mito-8-Ser is as follows: ¹H NMR (600 MHz, DMSO-d₆) δ7.87-7.93 (m, 3H), 7.75-7.85 (m, 11H), 7.65-7.74 (m, 1H), 7.54-7.58 (m, 1H), 7.18-7.33 (m, 3H), 6.87-7.09 (m, 3H), 4.29-4.36 (m, 1H), 3.54-3.63 (m, 2H), 2.70 (s, 2H), 2.30-2.47 (m, 2H), 2.17-2.28 (m, 1H), 1.96 (d, J=13.39 Hz, 1H), 1.61-1.72 (m, 2H), 1.54 (d, J=5.32 Hz, 4H), 1.41-1.50 (m, 2H), 1.38 (qd, J=7.41, 14.99 Hz, 1H), 1.21-1.32 (m, 7H); HRMS (ESI, m/z): calculated for C₄₄H₄₇Cl₂NOP⁺ [M]⁺706.2767, found 706.2758.

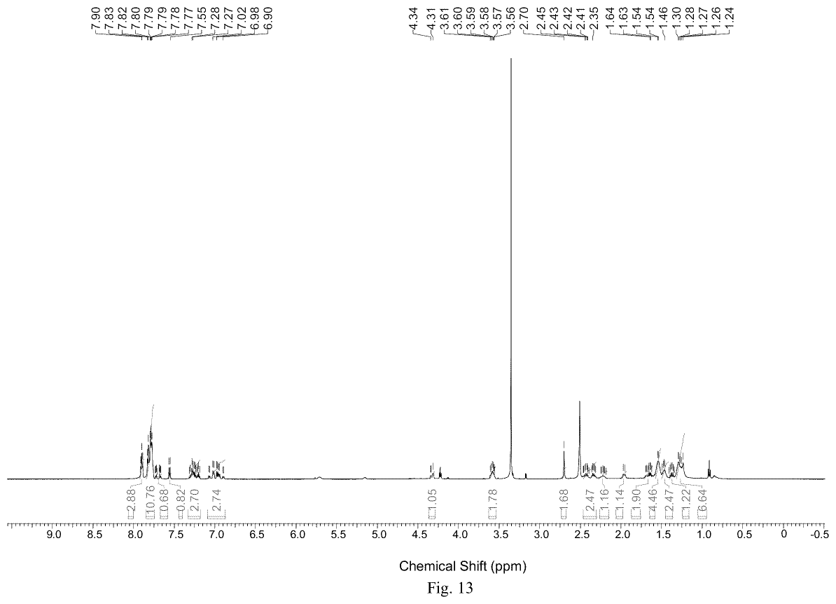

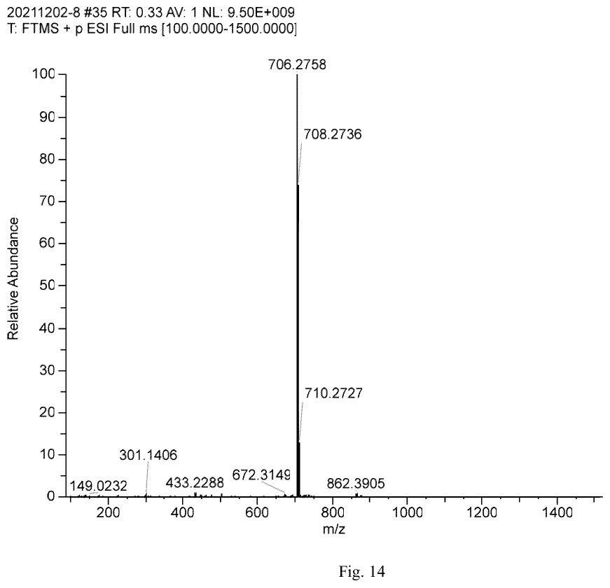

The hydrogen spectrum data of the Mito-8-Ser is shown in FIG. 13. The mass spectrum data of the Mito-8-Ser is shown in FIG. 14.

### Embodiment 5: Preparation of (11-(((1S,4S)-4-(3,4-dichlorophenyl)-1,2,3,4-tetrahydronaphthalen-1-yl)(methyl)amino)-11-oxoundecyl)triphenylphosphine (Mito-10-Ser)

The preparation method is the same as in Embodiment 1, 11-bromoundecanoic acid is used instead of 3-bromopropanoic acid in Embodiment 1, and the formula of the Mito-10-Ser is as follows:

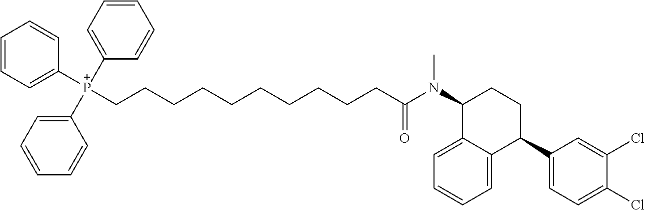 [SMILES: CN(C(=O)CCCCCCCCCC[P+](c1ccccc1)(c1ccccc1)c1ccccc1)[C@H]1CC[C@@H](c2ccc(Cl)c(Cl)c2)c2ccccc21]

The spectrum data of the Mito-10-Ser is as follows: ¹H NMR (600 MHz, DMSO-d₆) δ7.88-7.93 (m, 3H), 7.74-7.84 (m, 12H), 7.54-7.58 (m, 1H), 7.18-7.33 (m, 3H), 6.88-7.10 (m, 3H), 4.30-4.36 (m, 1H), 3.54-3.62 (m, 2H), 2.71 (s, 2H), 2.30-2.49 (m, 2H), 2.18-2.26 (m, 1H), 1.93-2.00 (m, 1H), 1.62-1.73 (m, 1H), 1.50-1.60 (m, 5H), 1.43-1.47 (m, 2H), 1.15-1.34 (m, 12H); HRMS (ESI, m/z): calculated for C₄₆H₅₁Cl₂NOP⁺ [M]⁺734.3080, found 734.3074.

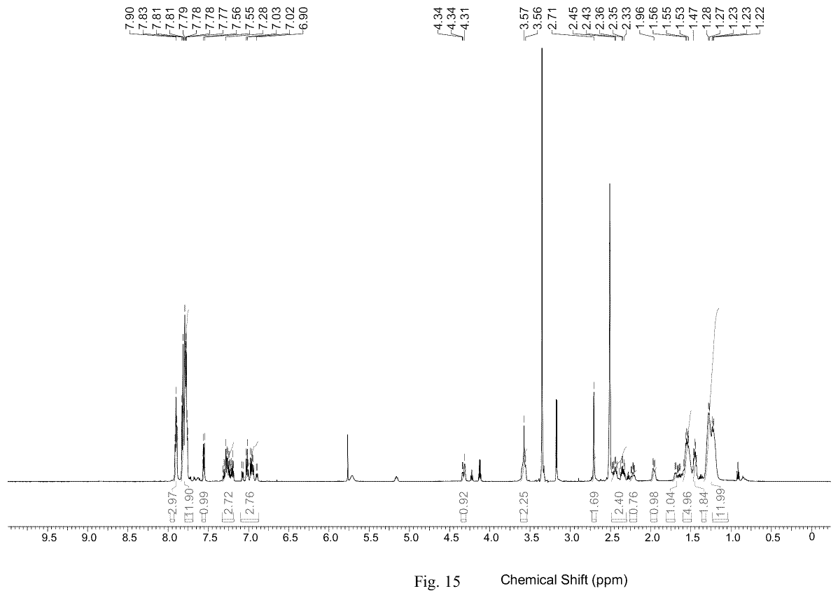

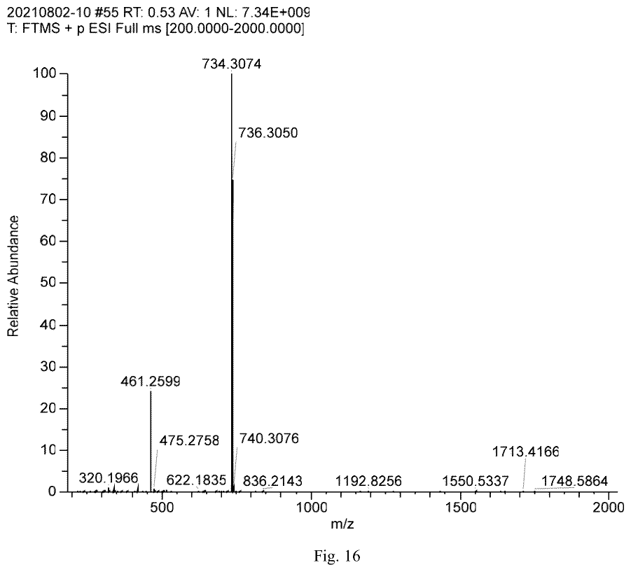

The hydrogen spectrum data of the Mito-10-Ser is shown in FIG. 15. The mass spectrum data of the Mito-10-Ser is shown in FIG. 16.

### Embodiment 6: Preparation of (13-(((1S,4S)-4-(3,4-dichlorophenyl)-1,2,3,4-tetrahydronaphthalen-1-yl)(methyl)amino)-13-oxytridecyl)triphenylphosphine (Mito-12-Ser)

The preparation method is the same as in Embodiment 1, 13-bromotridecanoic acid is used instead of 3-bromopropanoic acid in Embodiment 1, and the formula of the Mito-12-Ser is as follows:

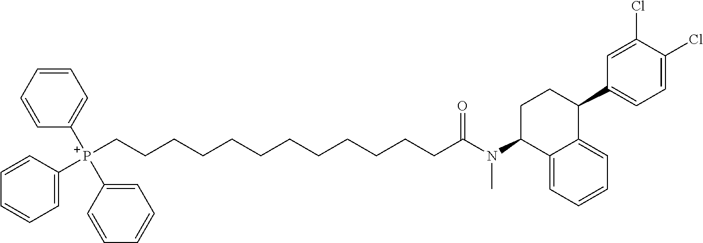 [SMILES: CN(C(=O)CCCCCCCCCCCC[P+](c1ccccc1)(c1ccccc1)c1ccccc1)[C@H]1CC[C@@H](c2ccc(Cl)c(Cl)c2)c2ccccc21]

The spectrum data of the Mito-12-Ser is as follows: ¹H NMR (600 MHz, DMSO-d₆) δ7.90 (d, J=6.42 Hz, 3H), 7.75-7.85 (m, 11H), 7.64-7.75 (m, 1H), 7.55 (d, J=8.07 Hz, 1H), 7.16-7.33 (m, 3H), 6.87-7.11 (m, 3H), 4.29-4.37 (m, 1H), 3.57 (br. s., 2H), 2.71 (br. s., 2H), 2.31-2.46 (m, 2H), 2.22 (br. s., 1H), 1.97 (br. s., 1H), 1.62-1.75 (m, 2H), 1.56 (br. s., 2H), 1.53 (br. s., 2H), 1.45 (br. s., 2H), 1.38 (dd, J=7.98, 15.31 Hz, 1H), 1.23 (br. s., 15H); HRMS (ESI, m/z): calculated for C₄₈H₅₅Cl₂NOP⁺ [M]⁺762.3393, found 762.3381.

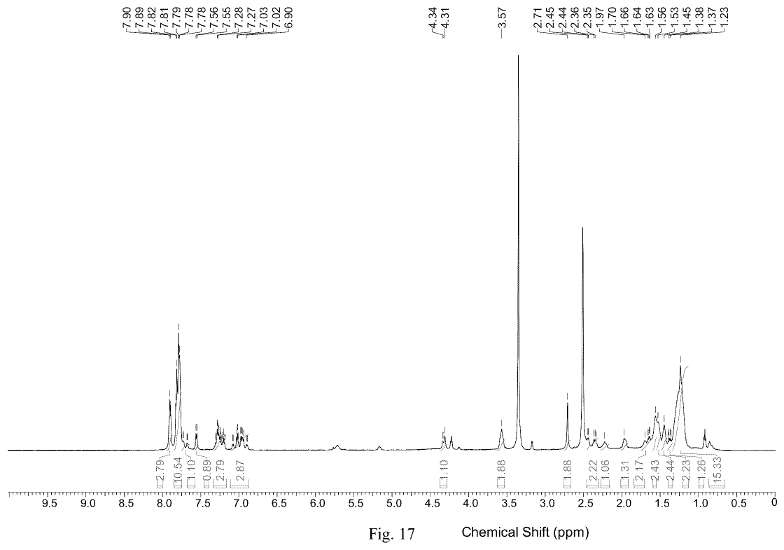

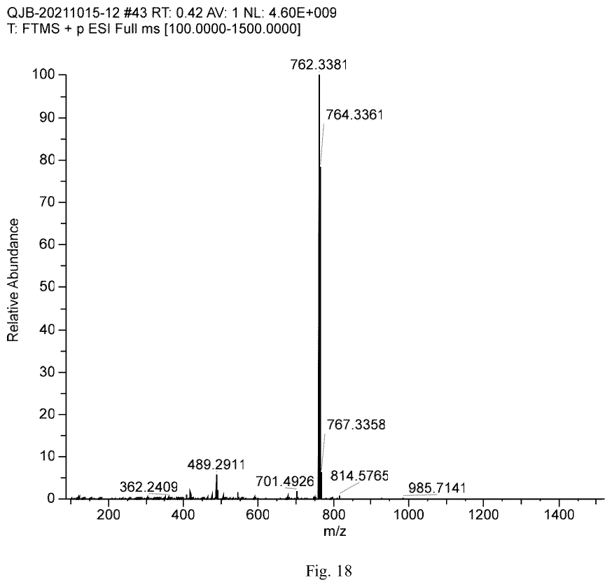

The hydrogen spectrum data of the Mito-12-Ser is shown in FIG. 17. The mass spectrum data of the Mito-12-Ser is shown in FIG. 18.

### Embodiment 7: Some Compounds of the Present Disclosure Induce Apoptosis in Human Non-Small Cell Lung Cancer Cells (A549 Cells) and Inhibit Mitochondria-Related Protein Expression

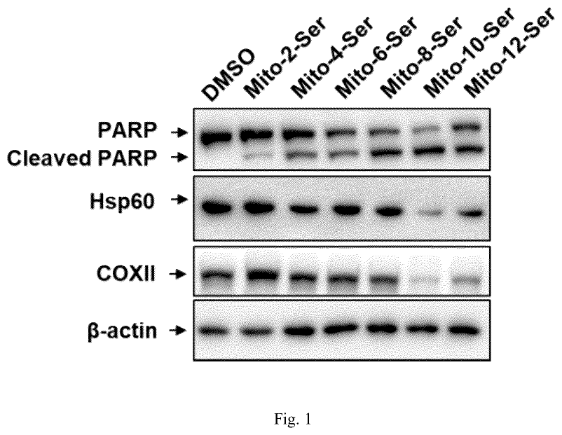

(1) Apparatus: PowerPac200 electrophoresis apparatus (Bio-Rad)
(2) Cell line: A549 (human non-small cell lung cancer cell line)
(3) Reagents: a. 2×SDS gel loading buffer: Tris-HCl (pH 8.0) (100 mM), β-mercaptoethanol (10%), glycerol (20%), bromophenol blue (0.2%), and sodium dodecyl sulfate (SDS) (4%). b. electrophoresis buffer: Tris (3.03 g), glycine (18.77 g), SDS (1 g), and deionized water (1 L). c. electrotransfer buffer: Tris (2.425 g), glycine (11.25 g), methanol (100 mL), and deionized water (1 L). d. Tris Buffered Saline with Tween-20 (TBST): Tris (1.21 g), NaCl (8.77 g), Tween-20 (1 mL), and deionized water (1 L). e. Developing solution (Guanlong), and stopping solution (Guanlong). f. Primary antibody: Hsp60 (Cell Signal Technology), COXII (Cell Signal Technology), Poly ADP-Ribose Polymerase (PARP) (Cell Signal Technology), and β-actin (proteintech). g. Secondary antibody: Horseradish Peroxidase (HRP)-coupled anti-mouse or anti-rabbit secondary antibodies
(4) Test method: cell lysates with the same amounts are boiled in sodium dodecyl sulfate (SDS) sample loading buffer, then separated, and electrotransferred to nitrocellulose membranes by 10% SDS-polyacrylamide gel electrophoresis (SDS-PAGE). The membranes are blocked using 5% skimmed milk in a buffer of the TBST (10 mM Trise-HCl [pH 8.0], 150 mM NaCl, and 0.05% Tween 20) at room temperature for 1 hour. The membranes are incubated with the specific primary antibody overnight at 4° C., washed, and then incubated with the HRP-coupled anti-mouse or anti-rabbit secondary antibodies, and immunoreaction products are observed by a chemiluminescence (ECL) solution. Meanwhile, the compound Mito-10-Ser is preferably selected. Activities of the compound Mito-10-Ser in inducing apoptosis and mitophagy in tumor cells are tested at different concentrations of 0.15 μM, 0.3 μM, 0.6 μM, 1.2 μM, 2.5 μM, and 5 μM, respectively. It is found that the compound Mito-10-Ser can induce increased expression of Cleaved PARP and decreased expression of Hsp60 and COXII in tumor cells (FIG. 1) by concentration gradients. It indicates that the Mito-10-Ser in the present disclosure has good activities in inducing the apoptosis and the mitophagy in the tumor cells.

### Embodiment 8: Flow Cytometry is Used to Demonstrate that Mito-IO-Ser Induces Apoptosis, Cell-Cycle Arrest, and Reduced Mitochondrial Membrane Potential in A549 Cells

(1) Cell apoptosis is tested using Annexin V-FITC/PI double staining
A. Apparatus: flow cytometer (BECKMAN COULTER)
B. Reagent: Annexin V-FITC/PI double staining apoptosis detection kit (BD)
C. Experimental method: cells are inoculated, and compounds dissolved in culture medium are added. After incubation for an appropriate time, A549 cells and A549 cisplatin-resistant cells are collected, added into pre-cooled 70% ethanol after removing the medium and washing by phosphate-buffered saline (PBS), and placed in a refrigerator at 4° C. overnight. The supernatant is removed, and the Annexin V-FITC is added and protected from light for 15 minutes. After that, propidium iodide (PI) dye with a same amount is then added into each tube and protected from light for 5 minutes. The samples are filtered using a 400-mesh sieve, transferred to flow detection tubes, and added into the flow cytometer for testing.
(2) Mitochondrial membrane potential damage is tested using JC-1
A. Apparatus: flow cytometer (BECKMAN COULTER)
B. Reagent: the JC-1 (BD)
C. Experimental method: A549 cells are incubated with the compounds for 12 hours. Cells are collected and washed with PBS after treatment and then stained with the JC-1 for 30 minutes at room temperature in the dark for flow cytometry analysis.
(3) The cell cycle is tested using PI single staining
A. Apparatus: flow cytometer (BECKMAN COULTER)
B. Reagent: Cell cycle analysis kit (Beyotime)

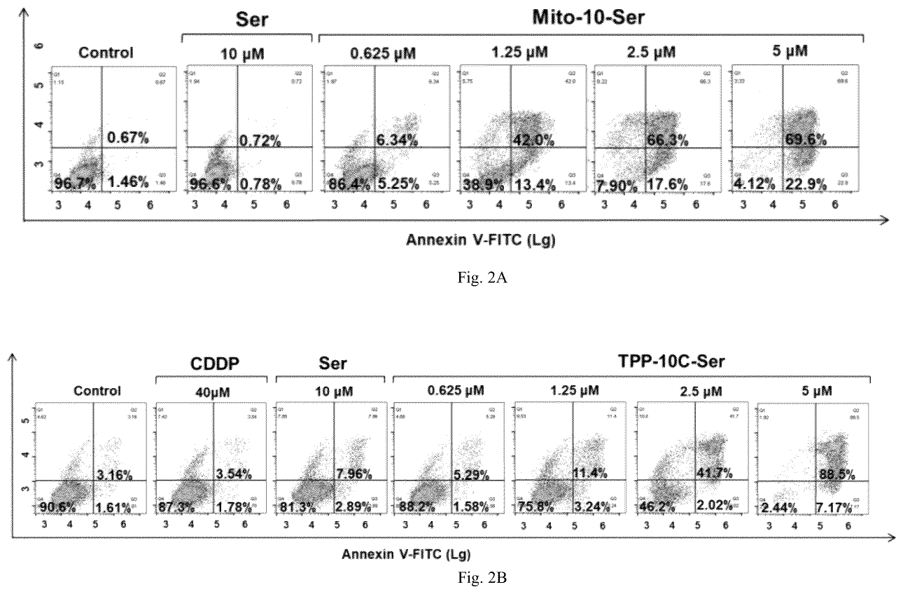

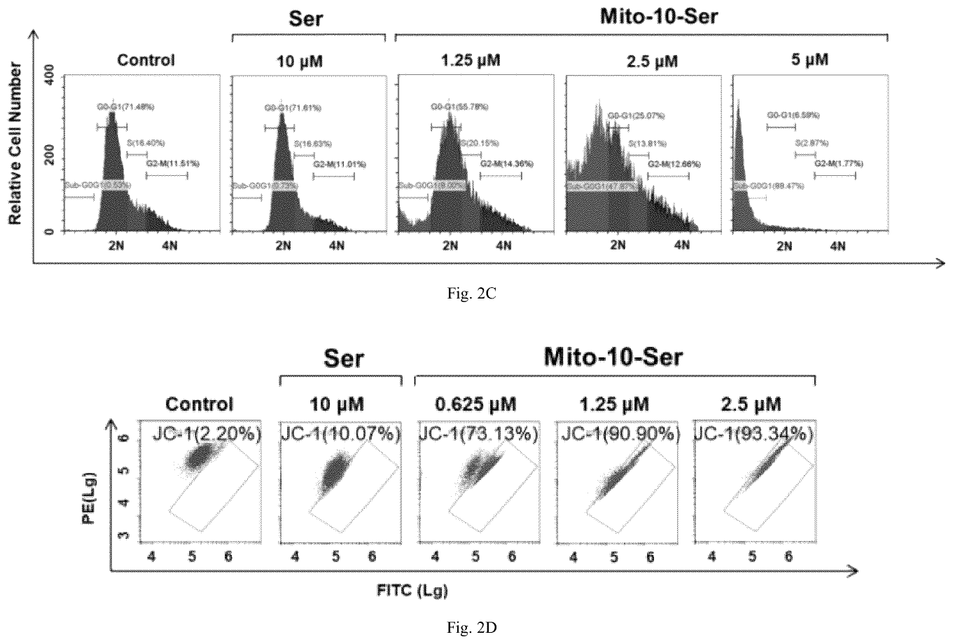

The experimental results show that the Mito-10-Ser can induce apoptosis of A549 cells and A549 cisplatin-resistant cells (FIGS. 2A and 2B) by concentration gradients and indicate that the Mito-10-Ser is equally effective on drug-resistant cells. Meanwhile, the Mito-10-Ser can induce cycle arrest (FIG. 2C) of tumor cells by concentration gradients and induce the decreased mitochondrial membrane potential (FIG. 2D) of the tumor cells (A549), thus inhibiting the proliferation of the tumor cells.

### Embodiment 9: Mito-2-Ser, Mito-10-Ser, and Mito-12-Ser can Induce Mitochondria Autophagy

(1) Apparatus: Transmission electron microscope Hitachi HT-7800
(2) Reagent: glutaraldehyde
(3) Experimental method: cells are inoculated in a 6 cm diameter Petri dish and incubated with the compounds for 3 hours. The culture solution is poured out; a 2 mL glutaraldehyde working solution (2.5% glutaraldehyde and phosphate buffer (PB) system) restored to room temperature is added and put into a refrigerator at 4° C. after fixation for 15 minutes at room temperature; the fixation solution is diluted 2-3 times with PB after fixation for 2-3 hours, the PB used for washing is poured off, PB containing 5% Bovine albumin (BSA) is added with an amount of 1 mL, and the adherent fixed cells are scraped off using a cell scraper, collected into 1.5 mL centrifuge tubes, and centrifuged for 4-5 minutes at 2,500 revolutions per minute (rpm) at 4° C. to prepare samples. The images are analyzed by a transmission electron microscope.

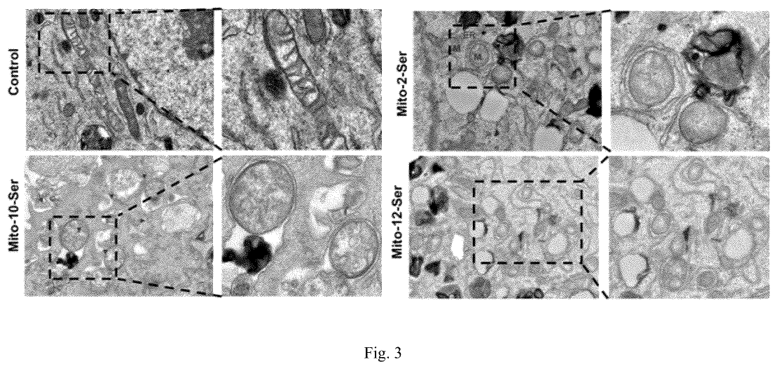

The experimental results show that the Mito-2-Ser, the Mito-10-Ser, and the Mito-12-Ser under a condition of a concentration of 0.3 μM and a work time of 3 hours induce autophagosome formation to phagocytize mitochondria and can rapidly induce interactions between lysosomes and endoplasmic reticulum and mitochondria in the tumor cells to promote the mitophagy (FIG. 3).

### Embodiment 10: Mito-10-Ser Inhibits Tumor Formation and Development in a Mouse Model of Lung Cancer (A549)

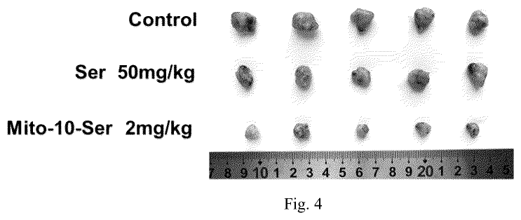

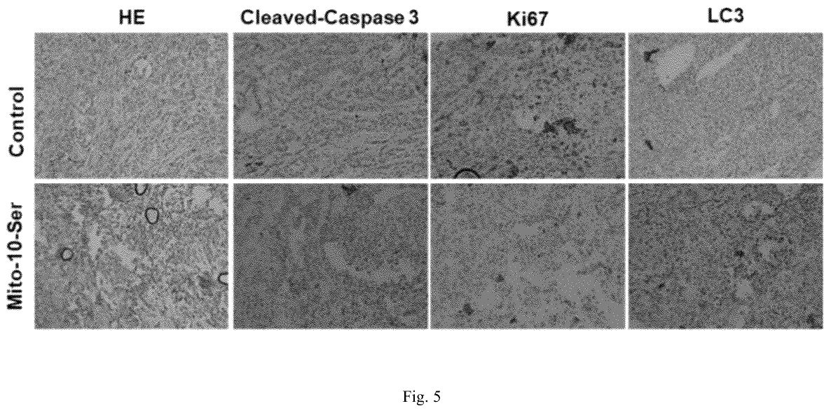

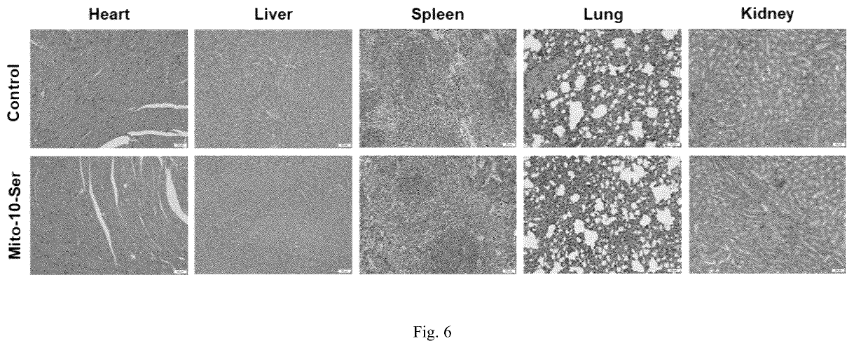

BALB/c (nu/nu) nude mice (with weights of 18-20 g and raised in an SPF-level animal room) are selected to construct a mouse transplantation tumor model in vitro, the mice are grouped and inoculated with human non-small cell lung cancer cells (A549), and tumor volumes in the mice grow to 100-300 mm³. The drug is administered by intraperitoneal injection, and each mouse in an administration group is administered once a day and 1 mg/kg once according to body weight. In a control group, each mouse is administrated with a same volume of PBS buffer, the volume of each mouse is measured simultaneously, and the drug is continuously administered for 15 days. After the drug administration and treatment process is complete, the mice are killed by cervical dislocation, and the mouse tumors are stripped, photographed, and weighed. The sizes and volumes of the tumors are countered, the experimental results shows that the Mito-10-Ser can well inhibit the human non-small cell lung cancer cells transplanted tumor model in mice (FIG. 4) in vivo and induce apoptosis and autophagy of the tumor tissue cells (FIG. 5). Immunohistochemical results of heart, liver, spleen, lung, and kidney in the administration group and the control group (FIG. 6) are tested to indicate that the Mito-10-Ser did not have significant toxic side effects.

The aforementioned description are merely preferred embodiments of the present disclosure, and the protective scope of the present disclosure is not limited thereto. It is intended that the present disclosure cover modifications and variations of the scope of the claims and the specification of the present disclosure.

1. A triphenylphosphine-modified sertraline derivative, wherein:
a structural formula is as follows: 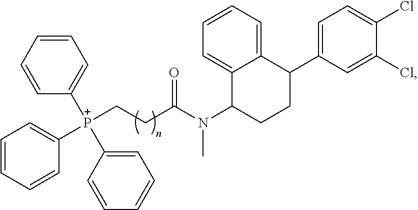
wherein n=1, 3, 5, 7, 9 or 11.

2. A preparation method of the triphenylphosphine-modified sertraline derivative according to claim 1, comprising:
(1) mixing p-triphenylphosphine, bromo organic acid, and a first organic solvent, and reacting by refluxing to obtain an intermediate, the bromo organic acid is 3-bromopropionic acid, 5-bromopentanoic acid, 7-bromoheptanoic acid, 9-bromononanoic acid, 11-bromoundecanoic acid, or 13-bromotridecanoic acid;
(2) mixing the intermediate obtained from the step (1), a second organic solvent, N-(3-dimethylaminopropyl)-N′-ethylcarbodiimide hydrochloride (EDCI), and 1-hydroxybenzotriazole (HOBt) to even under a room temperature condition, then adding sertraline hydrochloride and N,N-diisopropylethylamine, reacting at room temperature, then distillating under reduced pressure, and rotatably drying to obtain a crude product; and
(3) purifying the crude product obtained from the step (2) to obtain the triphenylphosphine-modified sertraline derivative.

3. A preparation method according to claim 2, wherein:
the first organic solvent is acetonitrile, and
the second organic solvent is dichloromethane.

4. An application of the triphenylphosphine-modified sertraline derivative according to claim 1 as an inducer for mitophagy.

5. An application of the triphenylphosphine-modified sertraline derivative according to claim 1 for preparing a drug for at least one of prevention or treatment of a mitophagy-related disease.

6. The application according to claim 5, wherein the mitophagy-related disease comprises tumors and neurodegenerative diseases.

7. A composition for at least one of prevention or treatment of a mitophagy-related disease, wherein:
an active ingredient of the composition comprises the triphenylphosphine-modified sertraline derivative according to claim 1 or a pharmaceutically acceptable salt, ester, prodrug, or hydrate thereof, and
the mitophagy-related disease comprises tumors and neurodegenerative diseases.

8. The composition according to claim 7, wherein:
the active ingredient is the triphenylphosphine-modified sertraline derivative or the pharmaceutically acceptable salt, ester, prodrug, or hydrate thereof.
